SEQUENCE LISTING (8 sequences)

SEQ ID NO: 1 - protein, 201 aa, Artificial
  PD-L2 extracellular domain
LFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPT

SEQ ID NO: 2 - protein, 124 aa, Artificial [sequence omitted]

SEQ ID NO: 3 - protein, 233 aa, Artificial
  Fc domain
QEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 15 aa, Artificial
  Linker
GGGGSGGGGSGGGGS

SEQ ID NO: 5 - protein, 573 aa, Artificial
  Mature fusion polypeptide
LFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTGGGGSGGGGSGGGGSMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - protein, 579 aa, Artificial
  Mature fusion polypeptide His tag
LFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTGGGGSGGGGSGGGGSMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 7 - protein, 598 aa, Artificial
  Fusion polypeptide precursor
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTGGGGSGGGGSGGGGSMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDQEPKSSDKTHTSPPSPAPELLGGSSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGKHHHHHH

SEQ ID NO: 8 - DNA, 1794 bp, Artificial
  Construct
atgattttcctgctgctgatgctgagcctggaactgcagctgcaccagattgccgcactgtttaccgtgaccgtccccaaagaactgtacatcatcgagcacggctctaacgtgaccctggagtgcaatttcgacacaggctcccatgtgaacctgggcgccatcaccgctagcctgcagaaggtggagaatgatacctctccacacagggagagggccacactgctggaggagcagctgccactgggcaaggcttcctttcatatccctcaggtgcaggtgagggacgagggccagtaccagtgcatcatcatctatggcgtggcctgggattacaagtatctgaccctgaaggtgaaggctagctaccggaagatcaacacccacatcctgaaggtgcccgagacagacgaggtggagctgacctgtcaggccacaggctatcctctggctgaggtgagctggcctaacgtgtctgtgccagccaatacctctcactccaggacacctgagggcctgtatcaggtgaccagcgtgctgaggctgaagccacctccaggacggaacttctcttgcgtgttttggaatacccatgtgagagagctgacactggcttccatcgatctgcagagccagatggagccacgcacccaccccacaggcggcggcggctctggaggaggaggctccggcggaggaggcagcatgcatgtggctcagccagctgtggtgctggcctccagcagaggcatcgcttccttcgtgtgcgagtacgcctctcccggcaaggctaccgaggtgagagtgacagtgctgaggcaggctgacagccaggtgaccgaggtgtgcgccgctacatatatgatgggcaacgagctgacctttctggacgattctatctgtaccggcacatcttccggcaaccaagtgaatctgaccatccagggcctgcgcgctatggatacaggcctgtacatctgcaaggtggagctgatgtatccccctccatactatctgggcatcggcaatggcacacagatctacgtgatcgacccagagccctgtcctgactctgatcaggagccaaagagctctgataagacccacacatccccacctagcccagctccagagctgctgggcggctccagcgtgttcctgtttccacccaagccaaaggacaccctgatgatctccaggacccccgaggtgacatgcgtggtggtggacgtgagccacgaggaccccgaggtgaagttcaactggtacgtggatggcgtggaggtgcataatgctaagacaaagccaagagaggagcagtacaactctacctatcgcgtggtgtccgtgctgacagtgctgcatcaggactggctgaacggcaaggagtataagtgcaaggtgtctaataaggccctgcctgctccaatcgagaagaccatctccaaggctaagggacagcccagggagcctcaggtgtacacactgcctccatcccgggacgagctgaccaagaaccaggtgagcctgacatgtctggtgaagggcttctatccttctgatatcgctgtggagtgggagtccaatggccagccagagaacaattacaagaccacaccccctgtgctggactctgatggctccttctttctgtattccaagctgaccgtggataagagcagatggcagcagggcaacgtgttctcctgttctgtgatgcacgaggccctgcataaccactacactcagaagtcactgtcactgtcaccaggaaagcaccatcatcatcatcat

(19) United States
(12) Patent Application Publication
(10) Pub. No.: US 2022/0089679 A1
(43) Pub. Date: Mar. 24, 2022

(54) FUSION PROTEIN WITH IMMUNOSUPPRESSIVE ACTIVITY

(71) Applicants: FUNDACIÓ INSTITUT D'INVESTIGACIÓ BIOMÈDICA DE BELL, L'Hospitalet de Llobregat (ES); UNIVERSITAT DE BARCELONA, Barcelona (ES)

(72) Inventors: Josep Maria GRINYO BOIRA, Barcelona (ES); Oriol BESTARD MATAMOROS, L'Hospitalet de Llobregat (ES); Juan TORRAS AMBROS, L'Hospitalet de Llobregat (ES)

(21) Appl. No.: 17/422,113

(22) PCT Filed: Jan. 10, 2020
(86) PCT No.: PCT/EP2020/050550

§ 371 Date: Jul. 9, 2021

(30) Foreign Application Priority Data
Jan. 11, 2019 (EP) 19382017.2

(51) Int. Cl. C07K 14/705; C12N 15/62

(52) U.S. Cl. CPC C07K 14/70532; C12N 15/62

(57) ABSTRACT

The present invention provides a compound comprising a fusion polypeptide of formula (I): R1-L-R2-Fc wherein R1, which is at the N-terminal end of the polypeptide, is PD-L2 or a PD1-binding fragment thereof, L is a peptide linker, R2 is CTLA-4 or a CD80/CD86-binding fragment thereof, and Fc, which is at the C-terminal end of the polypeptide, is an immunoglobulin Fc domain. The present invention also provides a dimer of the compound, a polynucleotide which encodes the polypeptide, a vector comprising the polynucleotide, a host cell which contains the polynucleotide, a composition and a kit comprising the compound. The invention also provides the compound or the dimer for use in therapy, diagnosis and prognosis, in particular for the treatment of autoimmune diseases or transplant rejection.

### CROSS REFERENCE TO RELATED APPLICATION

The present application is a 35 USC 371 national phase filing of PCT/EP2020/050550 filed on Jan. 10, 2020, which claims the benefit of and priority to European Patent Application EP19382017.2 filed on Jan. 11, 2019, both applications are incorporated herein by reference in their entirety.

### INCORPORATION BY REFERENCE

The present application contains a Sequence Listing which has been submitted in ASCII format via EFS-Web and is hereby incorporated by reference in its entirety. Said ASCII copy having been modified on Jul. 9, 2021, is named “108663_00271_SEQLISTING_ST25.txt” and is 23,280 bytes in size.

### TECHNICAL FIELD

The present invention belongs to the field of fusion proteins. In particular, the invention relates to a fusion protein with immunosuppressive activity. The fusion protein of the invention is particularly useful in the treatment of autoimmune diseases or in the treatment or prevention of transplant rejection.

### BACKGROUND ART

The human immune system protects the body from damage by foreign substances and pathogens. One way in which the immune system protects the body is by producing specialized cells, referred to as T lymphocytes or T cells. Intercellular interactions between T cells and antigen-presenting cells (APCs) generate T cell stimulatory signals that in turn lead to T cell responses to antigens. Full T cell activation requires Signal 1, which consists on the binding of the T cell receptor (TCR) to antigen-MHC complex present on antigen-presenting cells, and Signal 2, based on the binding of the receptor CD28 on the surface of the T cell to the CD86 and/or CD80 ligands present on the APCs. Without this second signal, Signal 1 leads the T cell anergic, unable to secrete cytokines and undergoes apoptosis. Signal 3 is mediated by the binding of secreted interleukins, mainly IL-2, to their cell surface receptors, which leads to T cell proliferation and clonal expansion.

Due to the crucial role that costimulatory signals play in T cell mediated immune responses, costimulatory pathways have been targeted to induce immunosuppression in conditions where the immune system acts in a non-beneficial manner to the organism. In the last two decades, several costimulatory pathways have been targeted using monoclonal antibodies or proteins. In fact, some of the agents developed for the inhibition of the CD80/86-CD28 and CD40-CD40L pathways have already entered in the clinic for the treatment of human autoimmune diseases or for the prevention of solid organ transplantation rejection.

For instance, soluble forms of CTLA4—an inhibitory effector of the CD80/86 pathway—have been constructed by fusing the variable-like extracellular domain of CTLA4 to immunoglobulin constant domains to provide CTLA4-Fc fusion proteins. Soluble CTLA4-Fc has been shown to prevent CD28-dependent co-stimulation by binding to both CD86 and CD80, and to inhibit co-stimulation of T cells and have beneficial immunosuppression effects in humans (Bruce S P. et al., “Update on abatacept: a selective co-stimulation modulator for rheumatoid arthritis”, Ann Pharmacother., 2007, vol. 41(7), pp. 1153-62). However, not all patients respond to CTLA4-Fc and continued response requires frequent drug administration, perhaps in part because blockade of interaction of CD28 with CD86/CD80 is a weak inducer of Tregs and insufficient for blocking activated effector T cell responses in a disease milieu.

The PD1-PD-L1/PD-L2 pathway is also under strong investigation for the development of immunosuppressive therapies. This costimulatory pathway consists of the programmed cell death-1 (PD-1) receptor and its ligands, PD-L1 and PD-L2, which deliver inhibitory signals that regulate the balance among T cell activation, tolerance, and immune-mediated tissue damage. Although most of the studies have been centered on the PD-L1 ligand, the higher affinity of PD-L2 and its expression pattern makes it a promising option for the development of immunomodulatory tools.

Attempts to improve the behavior of both CTLA-4 and PD-L2 have been reported in the prior art. For example, the international application WO201004105 discloses a fusion protein comprising, in N- to C-terminal direction, CTLA-4 and PD-L2. However, it was found that the resulting fusion protein had a significantly lower binding capacity to PD-1.

In spite of the efforts made so far in the field of immunotherapy, there is a lack of molecules to reliably and efficiently modulate immunosuppressive pathways.

### SUMMARY OF INVENTION

The present inventors have developed a novel fusion polypeptide comprising, in N- to C-terminal direction, domains of PD-L2, CTLA-4 and the constant region of an immunoglobulin. Surprisingly, the inventors found that this fusion polypeptide is capable of simultaneously target the CD80/86-CD28 and PD-1/PDL1-PDL2 immune pathways on T cells thereby strongly inhibiting T cell activation.

It is well-known in the state of the art that both PD-L2 and CTLA-4 display their binding domains in their N-terminal ends. Therefore these binding domains have to be free to appropriately bind to their respective targets, in order to provide the desired effect. In this regard, WO201004105 experimentally shows that a fusion polypeptide comprising CTLA-4-PDL-2 presents a significantly lower PD1 binding affinity due to the fact that PDL-2 binding sites are masked.

Before the present invention, therefore, the skilled person would have attempted to design the fusion polypeptide keeping the N-terminal end of both moieties free.

Unexpectedly, the inventors have found that the fusion polypeptide of the invention not only allowed a strong inhibition of T cell activation, but also, and more importantly, it generated a synergistic immunosuppressive effect on T cells that resulted in an inhibition even higher than the one produced by non-fused PD-L2 and CTLA-4. As it is shown in FIG. 5 the immunosuppressive effect of the fusion polypeptide of the invention was nearly complete, while the non-fused PD-L2/CTLA-4 administration only achieved about 75% reduction in T cell proliferation.

The remarkable improvement shown by the fusion polypeptide of the invention, comprising PD-L2 and CTLA-4, is indicative that it could be used for the treatment of immunosuppressive diseases, or even transplant rejection. Indeed, and as shown in examples below, the inventors found that the fusion polypeptide strongly improved the survival of murine models of lupus nephritis and renal transplantation.

In addition, the results reported herein with the fusion polypeptide can also be obtained when the fusion polypeptide forms part of a bigger compound (such as a protein).

In view of the above, the fusion polypeptide herein provided constitutes a great advance in the field of immunotherapy, and in particular for the treatment of autoimmune diseases and organ transplantation.

Thus, in a first aspect, the invention provides a compound comprising a fusion polypeptide of formula (I), wherein R1, which is at the N-terminal end of the polypeptide, is PD-L2 or a PD1-binding fragment thereof, L is a peptide linker, R2 is CTLA-4 or a CD80/CD86-binding fragment thereof, and Fc, which is at the C-terminal end of the polypeptide, is an immunoglobulin Fc domain.

R1-L-R2-Fc (I)

In a second aspect, the invention provides a dimer comprising two subunits, wherein one or both subunits correspond to the compound as defined in the first aspect.

In a third aspect, the invention provides a polynucleotide which encodes the compound as defined in the first aspect or the dimer as defined in the second aspect.

In a fourth aspect, the invention provides a vector comprising the polynucleotide as defined in the third aspect of the invention.

In a fifth aspect, the invention provides a host cell which is transformed or transfected with the polynucleotide as defined in in the third aspect of the invention or the vector as defined in in the fourth aspect.

In a sixth aspect, the invention provides a cell culture comprising the host cell as defined in the fifth aspect.

In a seventh aspect, the invention provides a process for the production of a compound as defined in the first aspect, comprising (a) culturing the host cell as defined in the fifth aspect; or, alternatively, (b) in vitro transcription and/or translation of the polynucleotide as defined in the third aspect; and (c) isolating the resulting compound.

In an eighth aspect the invention provides a process for the production of a dimer as defined in the second aspect of the invention comprising (a) culturing the host cell as defined in the fifth aspect; or, alternatively, (b) in vitro transcription and/or translation of the polynucleotide as defined in the third aspect; and (c) isolating the compound under non-reducing conditions.

The inventors have found that the resulting dimer, following the process of the eighth aspect of the invention, is characterized by the disulfide bond between the two subunits. Thus, the dimer of the second aspect of the invention can be formulated as the dimer obtainable by the process of the eighth aspect of the invention.

In a ninth aspect, the invention provides a pharmaceutical composition comprising a therapeutically effective amount of the compound as defined in the first aspect, or the dimer of the second aspect of the invention, with at least one pharmaceutically acceptable carrier or excipient.

In a tenth aspect, the invention provides a kit of parts comprising the compound as defined in the first aspect, or the dimer as defined in the second aspect of the invention, or the pharmaceutical composition as defined in the ninth aspect, and optionally, instructions for its use.

In an eleventh aspect, the invention provides the compound as defined in the first aspect, the dimer as defined in the second aspect, or the pharmaceutical composition as defined in the ninth aspect, for use in therapy, diagnosis or prognosis.

In a further aspect, the invention provides the compound as defined in the first aspect, the dimer as defined in the second aspect, or the pharmaceutical composition as defined in the ninth aspect, for use in therapy

In a further aspect, the invention provides the compound as defined in the first aspect, the dimer as defined in the second aspect, or the pharmaceutical composition as defined in the ninth aspect, for use in diagnosis or prognosis.

In a twelfth aspect, the invention also provides a method for inhibiting T cell activation, the method comprising the step of contacting an isolated biological sample of a subject, the compound as defined in the first aspect, the dimer as defined in the second aspect, or the pharmaceutical composition as defined in the ninth aspect. This aspect can alternatively be defined as the in vitro use of the compound as defined in the first aspect, the dimer as defined in the second aspect, or the pharmaceutical composition as defined in the ninth aspect in a method for inhibiting T cell activation in an isolated biological sample.

In a final aspect, the invention provides a method of transplantation of a mammalian organ or tissue, the method comprising removing the organ or tissue from a donor, contacting the organ or tissue with the compound as defined in the first aspect, the dimer as defined in the second aspect of the invention, or the pharmaceutical composition as defined in the ninth aspect, and transplanting said organ or tissue in the recipient.

### BRIEF DESCRIPTION OF DRAWINGS

FIG. 1, related to Example 1, shows the map of the expression vector pcDNA3.1 used for expressing the fusion polypeptide.

FIG. 2, related to Example 1, shows an SDSPAGE gel loaded with the cellular supernatant of the ExpiCHO cells expressing the fusion polypeptide of the invention at the culture time points indicated. The arrow indicates the band corresponding to the fusion polypeptide.

FIG. 3, related to Example 1, shows the chromatogram of the chromatography performed with culture supernatant (a) and Protein A column purification (b) of the polypeptide of the invention.

FIG. 4, related to Example 1, is a SDSPAGE gel loaded with the fusion polypeptide of the invention after purification using HiTrap Protein A HP column. The lower band corresponds to the monomer (reducing conditions) while the upper band corresponds to the dimer (non-reducing conditions).

FIG. 5, related to Example 2, shows the percentage of % CD3+ cells in a classical human mixed lymphocyte reaction (MLR) where cultures where treated with various stimuli. (A) shows the results of T cells from subject 51 and (B) shows the results of T cells from subject S2. “a” refers to untreated cells; “b” refers to cells treated with CTLA4-Fc (Abatacept) at 10 ug/ml; “c” refers to cells treated with PDL2-Fc at 10 ug/ml; “d” refers to cell simultaneously treated with CTLA4-Fc (Abatacept) at 10 ug/ml and PDL2-Fc at 10 ug/ml; “e” refers to cells treated with tacrolimus (TAC) at 10 ug/ml; “f” and “g” refer to cells treated with the fusion polypeptide of SEQ ID NO:6 at 10 ug/ml and 20 ug/ml, respectively; and “h” refers to cells treated with pokeweed, a mitogen agent, as positive control.

FIG. 6, related to Example 3, shows the survival curve of rats subjected to renal allotransplantation and treated with the fusion polypeptide of the invention or with PBS. The y-axis represents the cumulative survival of the animals. The x-axis represents the survival time in days.

FIG. 7, related to Example 4, shows various parameters of rats subjected to ischemia-reperfusion injury (IRI) and treated with the fusion polypeptide of the invention or left untreated. (A) shows the levels of serum creatinine (mg/dl) at the time points indicated (in days). “a” refers to non-treated rats (n=5); “b” refers to rats treated with the fusion polypeptide of the invention (Hybri); and “c” to sham operated rats (n=4). (B) shows the level of Tregs (number per high power field). “1” refers to non-treated rats, and “2” refers to rats treated with Hybri.

FIG. 8, related to Example 5, shows different parameters of NZB/W F1 hybrid mice that spontaneously develop lupus nephritis after treatment with the fusion polypeptide of the invention “B”, CYP “C”, or PBS “A”. (A) shows the albuminuria/creatininuria ratio with the various treatments at the different time points indicated. (B) shows the anti-dsDNA antibody levels in serum with the various treatments at the different time points indicated.

### DETAILED DESCRIPTION OF THE INVENTION

All terms as used herein in this application, unless otherwise stated, shall be understood in their ordinary meaning as known in the art. Other more specific definitions for certain terms as used in the present application are as set forth below and are intended to apply uniformly through-out the specification and claims unless an otherwise expressly set out definition provides a broader definition.

As mentioned above, the invention provides in a first aspect a compound comprising the fusion polypeptide of formula (I):

R1-L-R2-Fc (I)

wherein R1, which corresponds to the N-terminal of the polypeptide, is PD-L2 or a PD1-binding fragment thereof, L is a peptide linker, R2 is CTLA-4 or a CD80/CD86-binding fragment thereof, and Fc, which corresponds to the C-terminal of the polypeptide, is an immunoglobulin Fc domain.

In a particular embodiment of the first aspect, optionally in combination with any of the embodiments provided below, the compound is a protein. In a more particular embodiment, the protein is from 300 to 1500 amino acids in length. In a more particular embodiment, the protein is from 400 to 1000 amino acids in length. In a more particular embodiment, the protein is from 400 to 600 amino acids in length.

The term “PD-L2” as used herein refers to the protein named programmed cell death 1 ligand 2, B7DC, CD273, or PDCD1L2. It is formed by an extracellular domain, a transmembrane domain, and a cytoplasmic domain. The extracellular domain contains an Ig-like V-type domain and a Ig-like C2-type domain. The protein sequence from various species is available in several protein databases, such as Uniprot Q9BQ51_HUMAN Homo sapiens (Oct. 5, 2005 update); and Q9WUL5_MOUSE Mus musculus, (Jan. 11, 1999 update). A “PD1-binding fragment” of PD-L2 refers to any fragment of a PD-L2 protein capable of binding PD1. A way to test if a fragment maintains the ability of binding PD1 can be performed, for example, by mixed lymphocyte react (MLR) assays, as described in the examples bellow. Briefly, T cells from a subject are mixed with CD3⁺-depleted splenocytes and dendritic cells from another high HLA-mismatch subject, and the culture is treated either with the candidate PD1-binding fragment or mocked treated. Proliferation is measured by quantifying the levels of CD3⁺ cells, and proliferation levels are compared between the treated group and mocked treated group. If the levels of proliferating CD3⁺ cells are significantly lower in the group treated with the fragment, this will be indicative that the fragment maintains the ability of binding to PD1.

In a particular embodiment of the first aspect, optionally in combination with any of the embodiments provided above and below, R1 is the extracellular domain of PD-L2. In a more particular embodiment, PD-L2 is mammalian PD-L2, more particularly the extracellular domain of a mammalian PD-L2. In a more particular embodiment, PD-L2 is human PD-L2, more particularly the extracellular domain of a human PD-L2. In a more particular embodiment, the sequence of R1 has at least 85%, 86%, 87%, 88%, 89%, 90%, 91%, 92%, 93%, 94%, 95%, 96%, 97%, 98%, or 99% sequence identity with SEQ ID NO: 1. In a more particular embodiment, R1 is of sequence SEQ ID NO: 1.

In the present invention the term “identity” refers to the percentage of residues that are identical in the two sequences when the sequences are optimally aligned. If, in the optimal alignment, a position in a first sequence is occupied by the same amino acid residue as the corresponding position in the second sequence, the sequences exhibit identity with respect to that position. The level of identity between two sequences (or “percent sequence identity”) is measured as a ratio of the number of identical positions shared by the sequences with respect to the size of the sequences (i.e., percent sequence identity=(number of identical positions/total number of positions)×100).

A number of mathematical algorithms for rapidly obtaining the optimal alignment and calculating identity between two or more sequences are known and incorporated into a number of available software programs. Examples of such programs include the MATCH-BOX, MULTAIN, GCG, FASTA, and ROBUST programs for amino acid sequence analysis, among others. Preferred software analysis programs include the ALIGN, CLUSTAL W, and BLAST programs (e.g., BLAST 2.1, BL2SEQ, and later versions thereof).

For amino acid sequence analysis, a weight matrix, such as the BLOSUM matrixes (e.g., the BLOSUM45, BLOSUM50, BLOSUM62, and BLOSUM80 matrixes), Gonnet matrixes, or PAM matrixes (e.g., the PAM30, PAM70, PAM120, PAM160, PAM250, and PAM350 matrixes), are used in determining identity.

The BLAST programs provide analysis of at least two amino acid sequences, either by aligning a selected sequence against multiple sequences in a database (e.g., GenSeq), or, with BL2SEQ, between two selected sequences. BLAST programs are preferably modified by low complexity filtering programs such as the DUST or SEG programs, which are preferably integrated into the BLAST program operations. If gap existence costs (or gap scores) are used, the gap existence cost preferably is set between about −5 and −15. Similar gap parameters can be used with other programs as appropriate. The BLAST programs and principles underlying them are further described in, e.g., Altschul et al., “Basic local alignment search tool”, 1990, J. Mol. Biol, v. 215, pages 403-410.

For multiple sequence analysis, the CLUSTAL W program can be used. The CLUSTAL W program desirably is run using “dynamic” (versus “fast”) settings. Amino acid sequences are evaluated using a variable set of BLOSUM matrixes depending on the level of identity between the sequences. The CLUSTAL W program and underlying principles of operation are further described in, e.g., Higgins et al., “CLUSTAL V: improved software for multiple sequence alignment”, 1992, CABIOS, 8(2), pages 189-191.

The term “CTLA-4” as used herein refers to the protein named Cytotoxic T-lymphocyte protein 4, or CD152. It is formed by an extracellular domain Ig-like V-type domain, a transmembrane domain, and a cytoplasmic domain. The protein sequence from various species is available in several protein databases, such as Uniprot P16410_HUMAN Homo sapiens (Oct. 1, 2003 update); or P09793_MOUSE Mus musculus (Jan. 7, 1989 update). Human CTLA-4 is known to form homodimers through the cysteine 120 of its extracellular domain. A “CD80/86-binding fragment” of CTLA-4 refers to any fragment of a CTLA-4 protein capable of binding CD80/86. A way to test if a fragment maintains the ability of binding CD80/86 can be performed, for example, by mixed lymphocyte react (MLR) assays, as described in the examples bellow. Briefly, T cells from a subject are mixed with CD3⁺-depleted splenocytes and dendritic cells from another high HLA-mismatch subject, and the culture is treated either with the CD80/86-binding fragment or mocked treated. Proliferation is measured by quantifying the levels of CD3⁺ cells, and proliferations levels are compared between the group treated and mocked treated. If the level of proliferating CD3⁺ cells is significantly lower in the group treated with the fragment, this will be indicative that the fragment maintains the ability of binding to PD1.

In a particular embodiment of the first aspect, optionally in combination with any of the embodiments provided above and below, R2 is the extracellular domain of CTLA-4. In a more particular embodiment, CTLA-4 is mammalian CTLA-4, more particularly the extracellular domain of a mammalian CTLA-4. In a more particular embodiment, CTLA-4 is human CTLA-4, more particularly the extracellular domain of a human CTLA-4. In a more particular embodiment, the sequence of R2 has at least 85%, 86%, 87%, 88%, 89%, 90%, 91%, 92%, 93%, 94%, 95%, 96%, 97%, 98%, or 99% sequence identity with SEQ ID NO: 2. In a more particular embodiment, R2 is of sequence SEQ ID NO: 2.

As used herein, the “Fc region” contains the hinge region and the CH2 and CH3 constant domains of the heavy chain of an antibody. The term “hinge region” refers to the flexible region situated between the constant domains of the heavy chain CH1 and CH2. Antibodies may be of any class, such as IgG, IgA, or IgM; and of any subclass, such as IgG1 or IgG4.

In a particular embodiment of the first aspect, optionally in combination with any of the embodiments provided above and below, the immunoglobulin Fc domain comprises the hinge region, the CH2 domain, and the CH3 domain of an immunoglobulin. In a more particular embodiment, the immunoglobulin is human IgG. In a particular embodiment, the Fc sequence has at least 85%, 86%, 87%, 88%, 89%, 90%, 91%, 92%, 93%, 94%, 95%, 96%, 97%, 98%, 99% or 100% sequence identity with SEQ ID NO: 3. In a more particular embodiment, the Fc is of sequence SEQ ID NO: 3.

The term “peptide linker” refers to an amino acid sequence which joins and separates two polypeptide domains in a protein. In the context of the compound of the invention, the linker is the sequence which joins the R1 domain with the R2 domain of the polypeptide.

The linker of the compound of the invention allows joining the C-terminal end of PD-L2 to the N-terminal end of CTLA-4, without disrupting its binding ability to CD80/86.

In a particular embodiment of the first aspect, optionally in combination with any of the embodiments provided above and below, the peptide linker is from 5 to 50 amino acids in length. In a more particular embodiment, the peptide linker is from 10 to 20 amino acids in length. In a more particular embodiment, the peptide linker is of 15 amino acids in length.

In a particular embodiment, optionally in combination with any of the embodiments provided above and below, the peptide linker comprises non-polar amino acids and polar neutral amino acids. In a more particular embodiment, the peptide linker comprises serine and glycine residues. In a more particular embodiment, at least 50%, at least 60%, at least 70%, at least 80%, or at least 90% of the amino acids forming the peptide linker are selected from non-polar amino acids and polar neutral amino acids. In a more particular embodiment, at least 50%, at least 60%, at least 70%, at least 80%, or at least 90% of the amino acids forming the peptide linker are non-polar amino acids and polar neutral amino acids. In a more particular embodiment, at least 50%, at least 60%, at least 70%, at least 80%, or at least 90% of the amino acids of the peptide linker are glycine and serine. In a more particular embodiment, the peptide linker consists of non-polar amino acids and polar neutral amino acids. In a more particular embodiment, the peptide linker consists of Gly and one or more polar neutral amino acids. In a more particular embodiment, the peptide linker consists of Gly and a polar neutral amino acid. In a more particular embodiment, the peptide linker consists of one or more non-polar amino acids and Ser. In a more particular embodiment, the peptide linker consists of a non-polar amino acid and Ser. In a more particular embodiment, the peptide linker consists of serine and glycine residues. In a more particular embodiment, the peptide linker consists of serine and glycine residues, wherein the percentage of Ser residues, with respect to the total number of residues forming the linker, is in the range from 10 to 40%, from 15 to 35% or 20%.

In a particular embodiment, optionally in combination with any of the embodiments provided above and below, the sequence of the linker has a sequence identity of 85%, 86%, 87%, 88%, 89%, 90%, 91%, 92%, 93%, 94%, 95%, 96%, 97%, 98%, 99% or 100% with SEQ ID NO: 4. In a particular embodiment, the sequence of the linker consists of SEQ ID NO: 4.

In the present invention, the term “amino acid” refers to a molecule containing both an amino group and a carboxyl group. Amino acids can be classified by the side chain group. There are basically four different classes of amino acids determined by different side chains: (1) non-polar, (2) polar and neutral, (3) acidic and polar, (4) basic and polar.

Non-polar amino acids have side chains which are hydrocarbon alkyl groups (alkane branches) or aromatic (benzene rings) or heteroaromatic (e.g. indole ring). Illustrative non-limitative examples of common non-polar amino acids are Ala, Val, Leu, Ile, Pro, Trp, Gly, Phe, and Met.

Polar-neutral amino acids have polar but not charged groups at neutral pH in the side chain (such as hydroxyl, amide or thiol groups). Illustrative non-limitative examples of polar neutral amino acids are Ser, Thr, Cys, Tyr, Asn, and Gln.

Acid amino acids (hereinafter also referred as “acid and polar amino acid”) have acidic side chains at neutral pH. These are aspartic acid or aspartate (Asp) and glutamic acid or glutamate (Glu), among others. Their side chains have carboxylic acid groups whose pKa's are low enough to lose protons, becoming negatively charged in the process.

Basic amino acids (hereinafter also referred as “basic and polar amino acid”) have side chains containing nitrogen and resemble ammonia which is a base (such as amines, guanidines, or imidazole). Their pKa's are high enough that they tend to bind protons, gaining a positive charge in the process. Illustrative non-limitative examples of basic amino acids are Lys, Arg, and His.

The term “unnatural amino acid” comprises D-isomers of the 20 common naturally occurring alpha-amino acids or amino acids of formula (A)

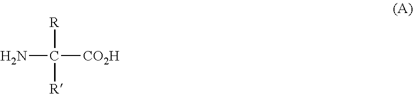

wherein R and R′ have the meaning provided in Table 1 below. Further illustrative non-limitative examples of unnatural amino acids are summarized in Table 2:

TABLE 1

| Exemplary unnatural | Suitable amino acid side chains | |
|---|---|---|
| alpha-amino acids | R | R′ |
| D-Alanine | —H | —CH₃ |
| D-Arginine | —H | —CH₂CH₂CH₂—NHC(═NH)NH₂ |
| D-Asparagine | —H | —CH₂C(═O)NH₂ |
| D-Aspartic acid | —H | —CH₂CO₂H |
| D-Cysteine | —H | —CH₂SH |
| D-Glutamic acid | —H | —CH₂CH₂CO₂H |
| D-Glutamine | —H | —CH₂CH₂C(═O)NH₂ |
| D-Histidine | —H | —CH₂-2-(1H-imidazole) |
| D-Isoleucine | —H | -sec-butyl |
| D-Leucine | —H | -iso-butyl |
| D-Lysine | —H | —CH₂CH₂CH₂CH₂NH₂ |
| D-Methionine | —H | —CH₂CH₂SCH₃ |
| D-Phenylalanine | —H | —CH₂Ph |
| D-Proline | —H | -2-(pyrrolidine) |
| D-Serine | —H | —CH₂OH |
| D-Threonine | —H | —CH₂CH(OH)(CH₃) |
| D-Tryptophan | —H | —CH₂-3-(1H-indole) |
| D-Tyrosine | —H | —CH₂-(p-hydroxyphenyl) |
| D-Valine | —H | -isopropyl |
| Di-vinyl | —CH═CH₂ | —CH═CH₂ |

| Exemplary unnatural | | |
|---|---|---|
| alpha-amino acids | | R and R′ are equal to: |
| α-methyl-Alanine (Aib) | —CH₃ | —CH₃ |
| α-methyl-Arginine | —CH₃ | —CH₂CH₂CH₂—NHC(═NH)NH₂ |
| α-methyl-Asparagine | —CH₃ | —CH₂C(═O)NH₂ |
| α-methyl-Aspartic acid | —CH₃ | —CH₂CO₂H |
| α-methyl-Cysteine | —CH₃ | —CH₂SH |
| α-methyl-Glutamic acid | —CH₃ | —CH₂CH₂CO₂H |
| α-methyl-Glutamine | —CH₃ | —CH₂CH₂C(═O)NH₂ |
| α-methyl-Histidine | —CH₃ | —CH₂-2-(1H-imidazole) |
| α-methyl-Isoleucine | —CH₃ | -sec-butyl |
| α-methyl-Leucine | —CH₃ | -iso-butyl |
| α-methyl-Lysine | —CH₃ | —CH₂CH₂CH₂CH₂NH₂ |

TABLE 2

| | |
|---|---|
| Aad | 2-Aminoadipic acid |
| bAad | 3-Aminoadipic acid |
| bAla | beta-Alanine, beta-Aminopropionic acid |
| Abu | 2-Aminobutyric acid |
| 4Abu | 4-Aminobutyric acid, piperidinic acid |
| Acp | 6-Aminocaproic acid |
| Ahe | 2-Aminoheptanoic acid |
| Aib | 2-Aminoisobutyric acid |
| bAib | 3-Aminoisobutyric acid |
| Apm | 2-Aminopimelic acid |
| Dbu | 2,4 Diaminobutyric acid |
| Des | Desmosine |
| Dpm | 2,2′-Diaminopimelic acid |
| Dpr | 2,3-Diaminopropionic acid |
| EtGly | N-Ethylglycine |
| EtAsn | N-Ethylasparagine |
| Hyl | Hydroxylysine |
| aHyl | allo-Hydroxylysine |
| 3Hyp | 3-Hydroxyproline |
| 4Hyp | 4-Hydroxyproline |
| Ide | Isodesmosine |
| alle | allo-Isoleucine |
| Nva | Norvaline |
| Nle | Norleucine |
| Orn | Ornithine |

Each one of the amino acids forming the peptide of the invention can have, independently from the others, L- or D-configuration.

Amino acids used in the preparation of the polypeptides of the present invention may be prepared by organic synthesis, or obtained by other routes, such as, for example, degradation of or isolation from a natural source.

In another embodiment of the first aspect of the invention, optionally in combination with any of the embodiments provided above or below, the compound consists of the fusion polypeptide of formula (I). In another embodiment of the first aspect of the invention, optionally in combination with any of the embodiments provided above or below, the compound consists of the fusion polypeptide of formula (I), and the N-terminal and C-terminal end corresponds to —NH2 and —COOH, respectively. In another embodiment of the first aspect of the invention, optionally in combination with any of the embodiments provided above or below, the compound consists of the fusion polypeptide of formula (I), and the C-terminal and N-terminal are derivatized. It is well-known in the state of the art how to derivatize the terminal ends of a peptide. In one embodiment, optionally in combination with any of the embodiments provided above or below, the C-terminal is amidated (—C(O)NH2). In one embodiment, optionally in combination with any of the embodiments provided above or below, the N-terminal is acetylated.

In another embodiment of the first aspect of the invention, optionally in combination with any of the embodiments provided above or below, the sequence of the fusion polypeptide has at least 85%, at least 90% or at least 95% identity with sequence SEQ ID NO: 5 or SEQ ID NO: 6; preferably a 100% of identity with sequence SEQ ID NO: 5 or SEQ ID NO: 6. In another embodiment of the first aspect of the invention, optionally in combination with any of the embodiments provided above or below, the compound consists of a fusion polypeptide having at least 85%, at least 90% or at least 95% identity with sequence SEQ ID NO: 5 or SEQ ID NO: 6; preferably a 100% of identity with sequence SEQ ID NO: 5 or SEQ ID NO: 6.

Sequences SEQ ID NO: 1 to 8 are summarized in Table 3 below:

TABLE 3

| SEQ ID | | |
|---|---|---|
| NO: | Sequence | Name |
| SEQ ID | LFTVTVPKELYIIEHGSNVTLECNFDTGSH | PD-L2 extracellular domain |
| NO: 1 | VNLGAITASLQKVENDTSPHRERATLLEE | |
| | QLPLGKASFHIPQVQVRDEGQYQCIIIYG | |
| | VAWDYKYLTLKVKASYRKINTHILKVPET | |
| | DEVELTCQATGYPLAEVSWPNVSVPANT | |
| | SHSRTPEGLYQVTSVLRLKPPPGRNFSC | |
| | VFWNTHVRELTLASIDLQSQMEPRTHPT | |
| SEQ ID | MHVAQPAVVLASSRGIASFVCEYASPGK | CTLA-4 extracellular |
| NO: 2 | ATEVRVTVLRQADSQVTEVCAATYMMG | domain |
| | NELTFLDDSICTGTSSGNQVNLTIQGLRA | |
| | MDTGLYICKVELMYPPPYYLGIGNGTQIY | |
| | VIDPEPCPDSD | |
| SEQ ID | QEPKSSDKTHTSPPSPAPELLGGSSVFL | Fc domain |
| NO: 3 | FPPKPKDTLMISRTPEVTCVVVDVSHEDP | |
| | EVKFNWYVDGVEVHNAKTKPREEQYNS | |
| | TYRVVSVLTVLHQDWLNGKEYKCKVSNK | |
| | ALPAPIEKTISKAKGQPREPQVYTLPPSR | |
| | DELTKNQVSLTCLVKGFYPSDIAVEWES | |
| | NGQPENNYKTTPPVLDSDGSFFLYSKLT | |
| | VDKSRWQQGNVFSCSVMHEALHNHYTQ | |
| | KSLSLSPGK | |
| SEQ ID | GGGGSGGGGSGGGGS | Linker |
| NO: 4 | | |
| SEQ ID | LFTVTVPKELYIIEHGSNVTLECNFDTGSH | Mature fusion polypeptide |
| NO: 5 | VNLGAITASLQKVENDTSPHRERATLLEE | |
| | QLPLGKASFHIPQVQVRDEGQYQCIIIYG | |
| | VAWDYKYLTLKVKASYRKINTHILKVPET | |
| | DEVELTCQATGYPLAEVSWPNVSVPANT | |
| | SHSRTPEGLYQVTSVLRLKPPPGRNFSC | |
| | VFWNTHVRELTLASIDLQSQMEPRTHPT | |
| | GGGGSGGGGSGGGGSMHVAQPAVVLA | |
| | SSRGIASFVCEYASPGKATEVRVTVLRQ | |
| | ADSQVTEVCAATYMMGNELTFLDDSICT | |
| | GTSSGNQVNLTIQGLRAMDTGLYICKVEL | |
| | MYPPPYYLGIGNGTQIYVIDPEPCPDSDQ | |
| | EPKSSDKTHTSPPSPAPELLGGSSVFLFP | |
| | PKPKDTLMISRTPEVTCVVVDVSHEDPEV | |
| | KFNWYVDGVEVHNAKTKPREEQYNSTY | |
| | RVVSVLTVLHQDWLNGKEYKCKVSNKAL | |
| | PAPIEKTISKAKGQPREPQVYTLPPSRDE | |
| | LTKNQVSLTCLVKGFYPSDIAVEWESNG | |
| | QPENNYKTTPPVLDSDGSFFLYSKLTVD | |
| | KSRWQQGNVFSCSVMHEALHNHYTQKS | |
| | LSLSPGK | |
| SEQ ID | LFTVTVPKELYIIEHGSNVTLECNFDTGSH | Mature fusion polypeptide |
| NO: 6 | VNLGAITASLQKVENDTSPHRERATLLEE | with poly His tag |
| | QLPLGKASFHIPQVQVRDEGQYQCIIIYG | |
| | VAWDYKYLTLKVKASYRKINTHILKVPET | |
| | DEVELTCQATGYPLAEVSWPNVSVPANT | |
| | SHSRTPEGLYQVTSVLRLKPPPGRNFSC | |
| | VFWNTHVRELTLASIDLQSQMEPRTHPT | |
| | GGGGSGGGGSGGGGSMHVAQPAVVLA | |
| | SSRGIASFVCEYASPGKATEVRVTVLRQ | |
| | ADSQVTEVCAATYMMGNELTFLDDSICT | |
| | GTSSGNQVNLTIQGLRAMDTGLYICKVEL | |
| | MYPPPYYLGIGNGTQIYVIDPEPCPDSDQ | |
| | EPKSSDKTHTSPPSPAPELLGGSSVFLFP | |
| | PKPKDTLMISRTPEVTCVVVDVSHEDPEV | |
| | KFNWYVDGVEVHNAKTKPREEQYNSTY | |
| | RVVSVLTVLHQDWLNGKEYKCKVSNKAL | |
| | PAPIEKTISKAKGQPREPQVYTLPPSRDE | |
| | LTKNQVSLTCLVKGFYPSDIAVEWESNG | |
| | QPENNYKTTPPVLDSDGSFFLYSKLTVD | |
| | KSRWQQGNVFSCSVMHEALHNHYTQKS | |
| | LSLSPGKHHHHHH | |
| SEQ ID | MIFLLLMLSLELQLHQIAALFTVTVPKELYI | Fusion polypeptide |
| NO: 7 | IEHGSNVTLECNFDTGSHVNLGAITASLQ | precursor with poly His tag |
| | KVENDTSPHRERATLLEEQLPLGKASFHI | |
| | PQVQVRDEGQYQCIIIYGVAWDYKYLTLK | |
| | VKASYRKINTHILKVPETDEVELTCQATG | |
| | YPLAEVSWPNVSVPANTSHSRTPEGLYQ | |
| | VTSVLRLKPPPGRNFSCVFWNTHVRELT | |
| | LASIDLQSQMEPRTHPTGGGGSGGGGS | |
| | GGGGSMHVAQPAVVLASSRGIASFVCEY | |
| | ASPGKATEVRVTVLRQADSQVTEVCAAT | |
| | YMMGNELTFLDDSICTGTSSGNQVNLTIQ | |
| | GLRAMDTGLYICKVELMYPPPYYLGIGNG | |
| | TQIYVIDPEPCPDSDQEPKSSDKTHTSPP | |
| | SPAPELLGGSSVFLFPPKPKDTLMISRTP | |
| | EVTCVVVDVSHEDPEVKFNWYVDGVEV | |
| | HNAKTKPREEQYNSTYRVVSVLTVLHQD | |
| | WLNGKEYKCKVSNKALPAPIEKTISKAKG | |
| | QPREPQVYTLPPSRDELTKNQVSLTCLV | |
| | KGFYPSDIAVEWESNGQPENNYKTTPPV | |
| | LDSDGSFFLYSKLTVDKSRWQQGNVFSC | |
| | SVMHEALHNHYTQKSLSLSPGKHHHHH | |
| | H | |
| SEQ ID | ATGATTTTCCTGCTGCTGATGCTGAGC | Polynucleotide encoding the |
| NO: 8 | CTGGAACTGCAGCTGCACCAGATTGCC | fusion polypeptide |
| | GCACTGTTTACCGTGACCGTCCCCAAA | precursor |
| | GAACTGTACATCATCGAGCACGGCTCT | |
| | AACGTGACCCTGGAGTGCAATTTCGAC | |
| | ACAGGCTCCCATGTGAACCTGGGCGC | |
| | CATCACCGCTAGCCTGCAGAAGGTGGA | |
| | GAATGATACCTCTCCACACAGGGAGAG | |
| | GGCCACACTGCTGGAGGAGCAGCTGC | |
| | CACTGGGCAAGGCTTCCTTTCATATCC | |
| | CTCAGGTGCAGGTGAGGGACGAGGGC | |
| | CAGTACCAGTGCATCATCATCTATGGC | |
| | GTGGCCTGGGATTACAAGTATCTGACC | |
| | CTGAAGGTGAAGGCTAGCTACCGGAAG | |
| | ATCAACACCCACATCCTGAAGGTGCCC | |
| | GAGACAGACGAGGTGGAGCTGACCTG | |
| | TCAGGCCACAGGCTATCCTCTGGCTGA | |
| | GGTGAGCTGGCCTAACGTGTCTGTGCC | |
| | AGCCAATACCTCTCACTCCAGGACACC | |
| | TGAGGGCCTGTATCAGGTGACCAGCGT | |
| | GCTGAGGCTGAAGCCACCTCCAGGAC | |
| | GGAACTTCTCTTGCGTGTTTTGGAATAC | |
| | CCATGTGAGAGAGCTGACACTGGCTTC | |
| | CATCGATCTGCAGAGCCAGATGGAGCC | |
| | ACGCACCCACCCCACAGGCGGCGGCG | |
| | GCTCTGGAGGAGGAGGCTCCGGCGGA | |
| | GGAGGCAGCATGCATGTGGCTCAGCC | |
| | AGCTGTGGTGCTGGCCTCCAGCAGAG | |
| | GCATCGCTTCCTTCGTGTGCGAGTACG | |
| | CCTCTCCCGGCAAGGCTACCGAGGTG | |
| | AGAGTGACAGTGCTGAGGCAGGCTGA | |
| | CAGCCAGGTGACCGAGGTGTGCGCCG | |
| | CTACATATATGATGGGCAACGAGCTGA | |
| | CCTTTCTGGACGATTCTATCTGTACCG | |
| | GCACATCTTCCGGCAACCAAGTGAATC | |
| | TGACCATCCAGGGCCTGCGCGCTATG | |
| | GATACAGGCCTGTACATCTGCAAGGTG | |
| | GAGCTGATGTATCCCCCTCCATACTAT | |
| | CTGGGCATCGGCAATGGCACACAGATC | |
| | TACGTGATCGACCCAGAGCCCTGTCCT | |
| | GACTCTGATCAGGAGCCAAAGAGCTCT | |
| | GATAAGACCCACACATCCCCACCTAGC | |
| | CCAGCTCCAGAGCTGCTGGGCGGCTC | |
| | CAGCGTGTTCCTGTTTCCACCCAAGCC | |
| | AAAGGACACCCTGATGATCTCCAGGAC | |
| | CCCCGAGGTGACATGCGTGGTGGTGG | |
| | ACGTGAGCCACGAGGACCCCGAGGTG | |
| | AAGTTCAACTGGTACGTGGATGGCGTG | |
| | GAGGTGCATAATGCTAAGACAAAGCCA | |
| | AGAGAGGAGCAGTACAACTCTACCTAT | |
| | CGCGTGGTGTCCGTGCTGACAGTGCT | |
| | GCATCAGGACTGGCTGAACGGCAAGG | |
| | AGTATAAGTGCAAGGTGTCTAATAAGG | |
| | CCCTGCCTGCTCCAATCGAGAAGACCA | |
| | TCTCCAAGGCTAAGGGACAGCCCAGG | |
| | GAGCCTCAGGTGTACACACTGCCTCCA | |
| | TCCCGGGACGAGCTGACCAAGAACCA | |
| | GGTGAGCCTGACATGTCTGGTGAAGG | |
| | GCTTCTATCCTTCTGATATCGCTGTGGA | |
| | GTGGGAGTCCAATGGCCAGCCAGAGA | |
| | ACAATTACAAGACCACACCCCCTGTGC | |
| | TGGACTCTGATGGCTCCTTCTTTCTGTA | |
| | TTCCAAGCTGACCGTGGATAAGAGCAG | |
| | ATGGCAGCAGGGCAACGTGTTCTCCTG | |
| | TTCTGTGATGCACGAGGCCCTGCATAA | |
| | CCACTACACTCAGAAGTCACTGTCACT | |
| | GTCACCAGGAAAGCACCATCATCATCA | |
| | TCAT | |

As mentioned above, in a second aspect, the invention provides a dimer comprising two subunits, wherein one or both subunits correspond(s) to the compound as defined in the first aspect.

All the embodiments of the compound of the first aspect are meant to apply to the dimer of the second aspect.

In a particular embodiment of the second aspect, optionally in combination with any of the embodiments provided above or below, the dimer is a homodimer or a heterodimer. In a more particular embodiment, the subunits forming the dimer are linked to each other by at least one disulfide bond. In a more particular embodiment, the subunits forming the dimer are linked to each other by one disulfide bond. In an even more particular embodiment, the disulfide bond is located between the two R2 domains. In an even more particular embodiment, the dimer is a homodimer wherein R2 corresponds to the extracellular domain of CTLA-4 (SEQ ID NO: 2) and the disulfide bond is between the cysteine residue at position 120 of each one of the CTLA-4 extracellular domain.

In a particular embodiment of the first or second aspects, optionally in combination with any of the embodiments provided above and below, the compound or the dimer further comprises a heterologous moiety.

As used herein, “heterologous moiety” refers to any molecule coupled to the fusion polypeptide via either a covalent or non-covalent bond. In a particular embodiment, the heterologous moiety is located in either the N-terminal or the C-terminal end of the compound. In a particular embodiment, the heterologous moiety is located in both the N-terminal and the C-terminal ends of the compound. The heterologous moiety can be, for example, a molecule that facilitates the purification of the protein. In a particular embodiment, the heterologous moiety is a peptide. In an even more particular embodiment, the heterologous moiety is a poly histidine track. In an even more particular embodiment, the heterologous moiety is a poly histidine track located in the C-terminal region of the compound or dimer. In a more particular embodiment, the poly histidine track consists of six histidines. As the skill in the art would understand, small peptides that assist in the purification of the protein can be maintained in the final compound without affecting its functionality.

Alternatively, the heterologous moiety can also be a diagnostic agent or a therapeutic agent.

In a more particular embodiment, the therapeutic agent is an immunosuppressive agent. A non-limiting list of immunosuppressive agents that can be used in the invention are methotrexate, dactinomycin, cyclosporin, 6-mercaptopurine, cyclophosphamide, mycophenolate, prednisolone, sirolimus, dexamethasone, rapamycin, FK506, mizoribine, azothioprine, tacrolimus, adalimumab, certolizumab, etanercept, golimumab, infliximab, belimumab, alefacept, abatacept, belatacept, tozcilizumab, and ustekinumab.

The immunosuppressive agent can be conjugated to the fusion polypeptide using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active asters (such as disuccinimidyl suberate), aldehydes (such as glutareldehyde), bis-azido compounds (such as bis (p-azidobenzoyl) hexanediamine), bis-diazonium derivatives (such as bis-(p-diazoniumbenzoyl)-ethylenediamine), diisocyanates (such as tolyene 2,6-diisocyanate), and bis-active fluorine compounds (such as 1,5-difluoro-2,4-dinitrobenzene).

In a more particular embodiment, the diagnostic agent is a label. The word “label” when used herein refers to a detectable compound or composition which is conjugated directly or indirectly to the fusion polypeptide so as to generate a “labelled” compound. The label may be detectable by itself (e.g. radioisotope labels or fluorescent labels) or, in the case of an enzymatic label, may catalyze chemical alteration of a substrate compound or composition which is detectable.

The label may be directly, attached or may be attached via a linker (such as Adipic Acid Dihyrazide (ADH). The label may be attached by chemical conjugation. Methods of conjugating labels to polypeptides are known in the art. For example, carbodiimide conjugation may be used to conjugate labels to antibodies. Other methods for conjugating a label to an antibody can also be used. For example, sodium periodate oxidation followed by reductive alkylation of appropriate reactants can be used, as can glutaraldehyde cross-linking.

The heterologous moiety can also be any vehiculization agent to facilitate the absorption, transport and delivery of the compound or dimer of the invention.

As mentioned above, in a third aspect, the invention provides a polynucleotide which encodes the compound as defined in the first aspect. In a particular embodiment of the third aspect, optionally in combination with any of the embodiments provided above and below, the polynucleotide is DNA or RNA.

As mentioned before, in a fourth aspect, the invention provides a vector comprising the polynucleotide of the third aspect. Examples of suitable vectors include those conventionally used in biomedicine and known to the skilled person.

As mentioned above, in a fifth aspect, the invention provides a host cell which is transformed or transfected with the polynucleotide or the vector of the invention. The skilled person would know which host cells are suitable for the synthesis of the protein of the invention.

In a particular embodiment of the fifth aspect, optionally in combination with any of the embodiments provided above and below, the host cell is a eukaryotic host cell. In a more particular embodiment the eukaryotic cell is selected from the group consisting of a CHO, HEK293 or Pichia pastoris cell. In an even more particular embodiment, the eukaryotic cell is an ExpiCHO cell.

In an alternative embodiment of the fifth aspect, optionally in combination with any of the embodiments provided above and below, the host cell is a prokaryotic host cell. In a more particular embodiment, the prokaryotic host cell is E. coli.

As mentioned before, in a sixth aspect the invention provides a cell culture comprising the host cell of the fifth aspect. Examples of suitable cell culture mediums and conditions include those conventionally used in cell biology and known to the skilled person.

As above mentioned, in a seventh aspect the invention provides process for the production of the compound according to the first aspect. The skilled person is familiar with several standard methods to isolate the resulting compound from the cell culture or after in vitro transcription and/or translation, for instance, Protein A purification column purification.

As above mentioned, an eighth aspect of the composition provides a process for the production of a dimer as defined in the second aspect of the invention comprising (a) culturing the host cell as defined in the fifth aspect; or (b) in vitro transcription and/or translation of the polynucleotide as defined in the third aspect; and (c) isolating the compound in non-reducing conditions.

As used herein, “non-reducing condition” refers to any condition that allows the formation of disulfide bonds between polypeptides or proteins. In a particular embodiment, the dimer is formed in an isotonic buffer with a pH similar to the isoelectric point of the fusion polypeptide. More in particular, the dimer is formed in a PBS buffer comprising 50 mM sodium phosphate, 150 mM NaCl, at pH 7. On the contrary, “reducing conditions” are those that do not allow the formation of disulfide bonds between polypeptides, such as the conditions provided by buffers that contain beta-mercaptoethanol or dithiothreitol (DTT). Therefore, in a particular embodiment, the monomer can be obtained in a buffer comprising 1 mM DTT.

Thus, the compound of the first aspect of the invention is a monomer in reducing conditions, and spontaneously dimerize in non-reducing conditions through the disulfide bonds located in the R2 domain.

As mentioned before, in a ninth aspect the invention provides a pharmaceutical composition comprising a therapeutically effective amount of the compound or the dimer of the invention, with at least one pharmaceutically acceptable excipient, diluent or carrier.

The expression “therapeutically effective amount” as used herein, refers to the amount of the compound or the dimer that, when administered, is sufficient to prevent development of, or alleviate to some extent, one or more of the symptoms of the disease or disorder which is addressed. The particular dose of agent administered according to this invention will of course be determined by the particular circumstances surrounding the case, including the compound or the dimer administered, the route of administration, the particular condition being treated, and the similar considerations.

The expression “pharmaceutical composition” encompasses both compositions intended for human as well as for non-human animals (i.e. veterinarian compositions).

The expression “pharmaceutically acceptable carriers or excipient” refers to pharmaceutically acceptable materials, compositions or vehicles. Each component must be pharmaceutically acceptable in the sense of being compatible with the other ingredients of the pharmaceutical composition. It must also be suitable for use in contact with the tissue or organ of humans and non-human animals without excessive toxicity, irritation, allergic response, immunogenicity or other problems or complications commensurate with a reasonable benefit/risk ratio.

Examples of suitable pharmaceutically acceptable excipients are solvents, dispersion media, diluents, or other liquid vehicles, dispersion or suspension aids, surface active agents, isotonic agents, thickening or emulsifying agents, preservatives, solid binders, lubricants and the like. Except insofar as any conventional excipient medium is incompatible with a substance or its derivatives, such as by producing any undesirable biological effect or otherwise interacting in a deleterious manner with any other component(s) of the pharmaceutical composition, its use is contemplated to be within the scope of this invention.

The relative amounts of the compound or the dimer, the pharmaceutically acceptable excipients, and/or any additional ingredients in a pharmaceutical composition of the invention will vary, depending upon the identity, size, and/or condition of the subject treated and further depending upon the route by which the composition is to be administered.

Pharmaceutically acceptable excipients used in the manufacture of pharmaceutical compositions include, but are not limited to, inert diluents, dispersing and/or granulating agents, surface active agents and/or emulsifiers, disintegrating agents, binding agents, preservatives, buffering agents, lubricating agents, and/or oils. Excipients such as coloring agents, coating agents, sweetening, and flavoring agents can be present in the composition, according to the judgment of the formulator.

The pharmaceutical compositions containing the compound or the dimer of the invention can be presented in any dosage form, for example, solid or liquid, and can be administered by any suitable route, for example, oral, parenteral, topical, intranasal or sublingual route, for which they will include the pharmaceutically acceptable excipients necessary for the formulation of the desired dosage form, for example, topical formulations (ointment, creams, lipogel, hydrogel, etc.), eye drops, aerosol sprays, injectable solutions, osmotic pumps, etc.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, corn-starch, powdered sugar, and combinations thereof.

Exemplary granulating and/or dispersing agents include, but are not limited to, potato starch, corn starch, tapioca starch, sodium starch glycolate, clays, alginic acid, guar gum, citrus pulp, agar, bentonite, cellulose and wood products, natural sponge, cation-exchange resins, calcium carbonate, silicates, sodium carbonate, cross-linked polyvinylpyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and combinations thereof.

Exemplary binding excipients include, but are not limited to, starch (e.g., corn-starch and starch paste); gelatin; sugars (e.g., sucrose, glucose, dextrose, dextrin, molasses, lactose, lactitol, mannitol); natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, polyvinylpyrrolidone), magnesium aluminium silicate (Veegum), and larch arabogalactan); alginates; polyethylene oxide; polyethylene glycol; inorganic calcium salts; silicic acid; polymethacrylates; waxes; water; alcohol; and combinations thereof.

Exemplary preservatives may include antioxidants, chelating agents, antimicrobial preservatives, antifungal preservatives, alcohol preservatives, acidic preservatives, and other preservatives. Exemplary antioxidants include, but are not limited to, alpha tocopherol, ascorbic acid, ascorbyl palmitate, ascorbyl stearate, ascorbyl oleate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite. Exemplary chelating agents include ethylenediaminetetraacetic acid (EDTA), citric acid monohydrate, disodium edetate, dipotassium edetate, edetic acid, fumaric acid, malic acid, phosphoric acid, sodium edetate, tartaric acid, and trisodium edetate.

Exemplary buffering agents include, but are not limited to, citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and combinations thereof.

Exemplary lubricating agents include, but are not limited to, magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and combinations thereof.

As above mentioned, in an eleventh aspect the invention provides the compound, the dimer, or the pharmaceutical composition of the invention for use in therapy, diagnosis or prognosis.

The inventors have demonstrated that the compound of the invention is highly useful in the treatment of various models of autoimmune disease and transplant rejection.

In a particular embodiment of the eleventh aspect, optionally in combination with any of the embodiments above or below, the compound, the dimer, or the pharmaceutical composition is for the treatment and/or prevention of a disease selected from an autoimmune disease and transplant rejection.

This embodiment can also be formulated as the use of the compound of the first aspect, the dimer of the second aspect, or the pharmaceutical composition of the ninth aspect for the manufacture of a medicament for the treatment and/or prevention of a disease selected from an autoimmune disease and transplant rejection. This aspect can also be formulated as a method for treating and/or preventing a disease selected from an autoimmune disease and transplant rejection, the method comprising administering a therapeutically effective amount of the compound of the first aspect, the dimer of the second aspect, or the pharmaceutical composition of the ninth aspect, to a subject in need thereof.

In a particular embodiment of the eleventh aspect, optionally in combination with any of the embodiments above or below, the autoimmune disease is selected from type 1 diabetes, Systemic Lupus Erythematosus, Rheumatoid Arthritis, inflammatory bowel disease, ulcerative colitis, Crohn's disease, multiple sclerosis, scleroderma, pemphigus vulgaris, psoriasis, atopic dermatitis, celiac disease, Chronic Obstructive Lung disease, Hashimoto's thyroiditis, Graves' disease, Sjögren's syndrome, Guillain-Barré syndrome, Goodpasture's syndrome, Addison's disease, autoimmune necrotising vasculitis (Wegener's granulomatosis), primary biliary sclerosis, sclerosing cholangitis, autoimmune hepatitis, polymyalgia rheumatica, Raynaud's phenomenon, temporal arteritis, giant cell arteritis, autoimmune hemolytic anemia, pernicious anemia, polyarteritis nodosa, Behcet's disease, primary biliary cirrhosis, uveitis, myocarditis, rheumatic fever, ankylosing spondylitis, glomerulonephritis, sarcoidosis, dermatomyositis, myasthenia gravis, polymyositis, alopecia areata, and vitiligo.

In a particular embodiment of the eleventh aspect, optionally in combination with any of the embodiments above or below, the transplant rejection is solid organ transplant rejection.

In a particular embodiment of the eleventh aspect, optionally in combination with any of the embodiments above or below, the compound, the dimer, or the pharmaceutical composition of the invention is for use in the diagnosis or prognosis of cancer.

In a particular embodiment of the eleventh aspect, optionally in combination with any of the embodiments above or below, the compound, the dimer, or the pharmaceutical composition of the invention is administered in combination, either sequentially or simultaneously, with an immune suppressant or modulator. A list of suitable immune suppressants or modulators has been provided above.

As mentioned above, in an twelfth aspect, the invention provides a method for inhibiting T cell activation comprising the step of contacting an isolated biological sample of a subject, the compound as defined in the first aspect, the dimer as defined in the second aspect, or the pharmaceutical composition as defined in the ninth aspect.

As used herein, the term “isolated biological sample” refers to any sample that contains T cells and that it is obtained from any tissue or fluid of a subject.

As used herein, the term “T cell” refers to cells of the immune system that contain the T cell receptor on their surface. They include CD4-positive (CD4⁺) helper T cells and CD8-positive (CD8⁺) cytotoxic T cells, where the former relates to promoting immune response and the latter relates to excluding virus-infected cells and tumor cells. “T cell activation” refers to the metabolic, morphological and functional changes that occur in a T cell that ensue upon antigen recognition. The activation is commonly accompanied by cell proliferation, cytokine secretion, differentiation into effector cells and memory cells.

The results herein provided demonstrate that the compound or dimer of the invention can be useful in reducing the rejection risk to an organ to be transplanted in a recipient by inhibiting T-cell activation. With this aim, the compound or dimer can be administered to the subject in the form of a pharmaceutical composition, as provided above but, in addition, or alternatively, the extracted organ, previous to the transplantation, can be subjected to a treatment with a sterile solution, comprising the compound or dimer of the invention.

As mentioned above, in a thirteenth aspect, the invention provides the in vitro use of the compound as defined in the first aspect, the dimer as defined in the second aspect, or the pharmaceutical composition as defined in the ninth aspect in a method for inhibiting T cell activation in an isolated biological sample.

In a particular embodiment of the twelfth and thirteenth aspects, optionally in combination with any of the embodiments above or below, the isolated biological sample is an organ or a tissue.

All the embodiments of the first aspect are also meant to apply to the seventh, eighth, ninth, tenth, eleventh, twelfth, and thirteenth aspects of the invention.

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples and drawings are provided by way of illustration, and they are not intended to be limiting of the present invention. Reference signs related to drawings and placed in parentheses in a claim, are solely for attempting to increase the intelligibility of the claim, and shall not be construed as limiting the scope of the claim. Furthermore, the present invention covers all possible combinations of particular and preferred embodiments described herein.

### EXAMPLES

### Example 1: Production and Purification of PDL2-CTLA4-Fc Fusion Protein

Design of Fusion Protein and Gene Synthesis

A fusion protein comprising human PDL2 extracellular domain, human CTLA4 extracellular domain and the Fc of human IgG was synthesized.

First the sequence of the protein of interest was designed (SEQ ID NO: 7, above disclosed). Amino acids 1 to 19 correspond to human PDL2 signal peptide, 20 to 220 correspond to mature human PDL2 extracellular domain. Amino acids 221 to 235 correspond to a (GGGGS)₃ linker. Amino acid 236 to 359 correspond to human CTLA4 extracellular domain, amino acids 360 to 592 correspond to the Fc of human IgG, and amino acids 593 to 598 correspond to a poly-histidine tag (6 histidines).

The precursor of the fusion protein (SEQ ID NO: 7) contains a PDL2 signal peptide that is not present in the mature form of the protein (SEQ ID NO: 6).

The amino acid sequence was converted to DNA and synthetized by the company Genscript (SEQ ID NO: 8, above described).

The gene of the fusion protein was cloned in pcDNA™3.1⁽⁺⁾ (ThermoFisher Scientific, V79020) expression vector for transfection and expression on CHO cells. The vector map of pcDNA3.1 is showed in FIG. 1.

Transfection and Cell Culture

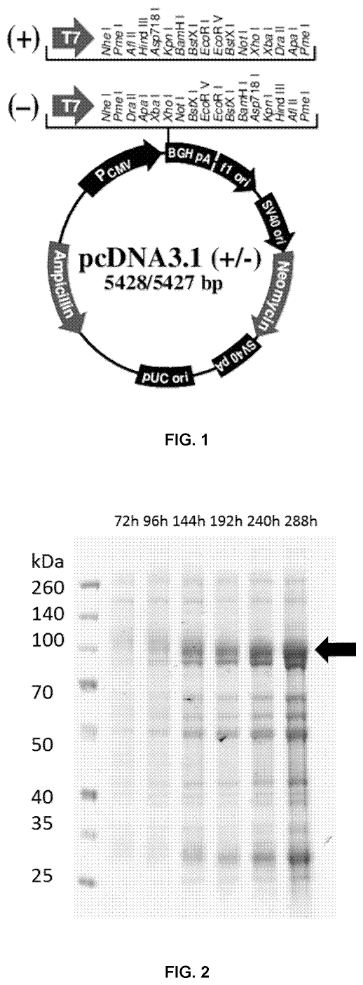

Transfection of ExpiCHO-S™ cells (ThermoFisher Scientific, A29127) was performed at a volume of 0.5 liters in serum free ExpiCHO™ Expression medium (ThermoFisher Scientific, A2910004). Transfection was carried out with a DNA amount of 0.5 mg and a Expifectamine concentration of 1.25 mL/mL following manufacturer's instructions. The cells were incubated for 14 days at 32° C. in an orbital shaker incubator. After 14 days the culture was harvested and cells were separated from supernatant by centrifugation. The production of the fusion protein was analyzed by SDSPAGE (Laemmli, U. K. “Cleavage of Structural Proteins during the Assembly of the Head of Bacteriophage T4”, Nature, 1970, vol. 227(5259), pp. 680-685). FIG. 2 shows the production of the fusion protein along the culture time.

Protein Purification

The fusion protein was purified by affinity chromatography using a 1 mL HiTrap Protein A HP column (GE Healthcare) following manufacturer's instructions. A volume of 650 mL of culture supernatant was loaded in the chromatographic column after centrifugation at 10.000 g for 15 minutes.

Binding buffer was PBS and elution buffer was 50 mM sodium phosphate, 150 mM NaCl, pH 2.5. Elution was carried out with 100% of elution buffer. Elution fractions were adjusted to pH 7.0 by adding 30 μL of 1M Hepes, pH 9.0 to 0.5 mL fractions.

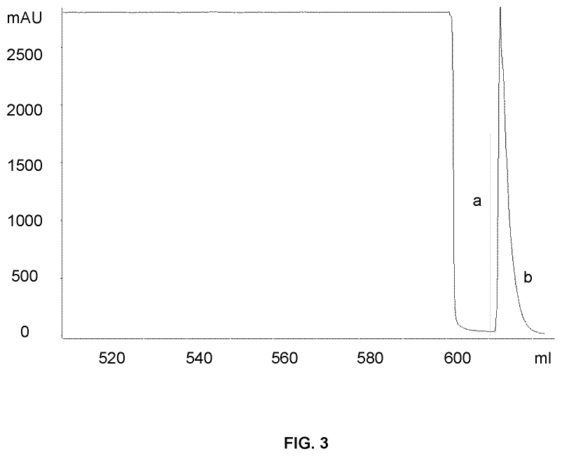

The chromatogram of FIG. 3 shows the affinity chromatography performed with culture supernatant and Protein A column.

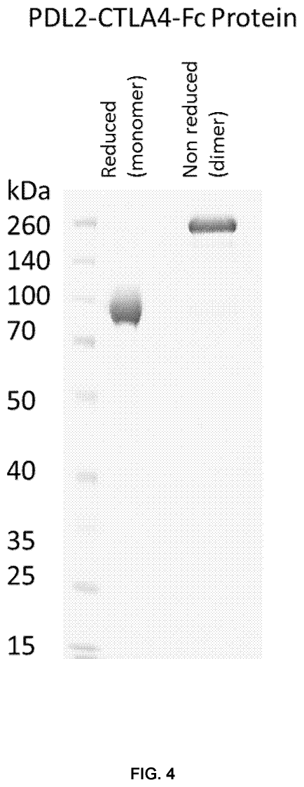

The purity of the purified fusion protein was analyzed by SDSPAGE. Also, the formation of dimers was analyzed by running the sample under non-reducing conditions. As can be observed in FIG. 4, the protein is highly purified and it is in dimeric form. The observed molecular weight of the dimer is 172 kDa (calculated from monomer mobility in SDSPAGE).

After chromatography the protein sample was filtered through a 0.22 μm porous size filter and stored in buffer HEPES 60 mM, sodium phosphate 47 mM, NaCl 140 mM, pH 7.2. The protein quantification was carried out by UV absorbance. The final volume of sample was 26 mL and the final concentration 0.35 mg/mL, in total 9.1 mg of protein were purified. From a total of 26 vials, 20 vials were lyophilized and 6 vials were stored at −20° C.

The data above demonstrates the purity of the fusion protein, and it also indicates that the poly-Histidine track is completely dispensable for the purification of the protein.

### Example 2: In Vitro Proliferation Assays

Mixed Lymphocyte reactions (MLR) were performed in the presence or absence of different immunosuppressive drugs to test their inhibitory effect on T cell proliferation, following conventional methodology (Levitsky J, et al., “Allospecific regulatory effects of sirolimus and tacrolimus in the human mixed lymphocyte reaction”, Transplantation, 2011, vol. 91(2), pp. 199-206).

Briefly, peripheral blood mononuclear cells (PBMC) from healthy controls were used as responder cells. CD3⁺-depleted splenocytes extracted from deceased donors and mature dendritic cells (DCs) were extracted with CD2 positive selection cocktail (Stem Cell Technologies, France) following manufacturer's instructions and they were used as stimulators in the assay. PBMC from peripheral blood samples and splenocytes were isolated by standard Ficoll density gradient centrifugation (Bargalló M E. et al., “Utility of Systematic Isolation of immune cell subsets from HIV-infected individuals for miRNA profiling”, J Immunol Methods, 2017, vol. 442, pp. 12-19). In order to obtain mature DCs, monocytes were isolated from PBMC by negative selection with Human monocyte enrichment kit (Stem Cell Technologies, France) following manufacturer's instructions, cultured for 6 days with complete Ex-vivo medium supplemented with 2% of human serum (Sigma Aldrich), GMSF (15 ng/ml; R&D) and IL-4 (10 ng/ml; Sigma Aldrich) (37° C. 5% CO₂), and stimulated with LPS (1 μg/ml; Sigma Aldrich) for 24 h.

First, PBMC were stained with carboxyfluorescein succinimidyl ester (CFSE) (Lloberas N, et al., “Dendritic cells phenotype fitting under hypoxia or lipopolysaccharide; adenosine 5′-triphosphate-binding cassette transporters far beyond an efflux pump”, Clin Exp Immunol., 2013, vol. 172(3), pp. 444-54). Then cells were co-cultured (2×10⁵ cells/100 μl) with splenocytes (1:1 ratio) or either mature DCs (10:1 ratio) in the presence of PD-L2-Ig (10 μg/mL; AB Biosciences) (Latchman Y, et al. “PD-L2 is a second ligand for PD-1 and inhibits T cell activation”, Nat Immunol., 2001, vol. 2(3), pp. 261-8), CTLA4-Ig (Abatacept) (10 μg/mL), the combination of both at the same concentrations in the media, tacrolimus (10 nM), the fusion protein of the invention of sequence SEQ ID NO: 6 (Hybri) at 10 μg/mL and 20 μg/mL, complete medium as a negative control for 6 days (37° C. 5% CO₂), and pokeewed mitogen (1 μg/mL), as an unspecific stimulator of T cells. At day 6, cells were harvested and stained for CD3-APC and CD4-PE (BD Bioscience, San Jose, Calif., USA) 20 minutes in the dark following manufacturer's instructions, and T-cell proliferation were analyzed by flow cytometry (BD FACS CANTO 11, San Jose, Calif., USA) using Facs Diva software and following manufacturer's instructions.

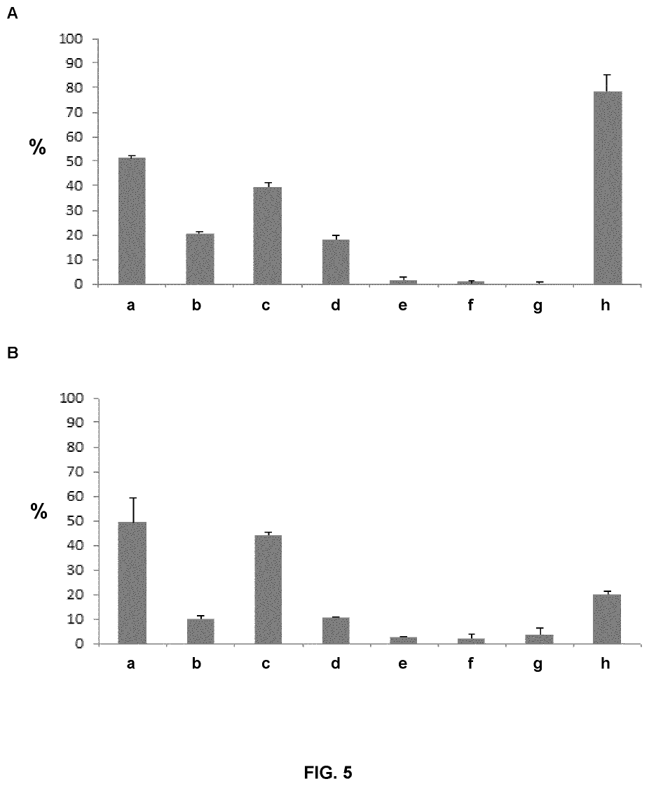

The in vitro proliferation assay was performed with high HLA-mismatch combination between 2 stimulator DCs and two corresponding responder subjects (51, S2) T cells. Hybri, at the two different concentrations indicated, was able to strongly inhibit mature CD3⁺ T cell proliferation to similar levels than tacrolimus (FIG. 5), which is considered a strong inhibitor of alloreactive T cell responses. CTLA4-Ig reduced by 40%-80% CD3⁺ cells proliferation as compared to spontaneous proliferation without immunosuppressive agents (MLR), PD-L2-Ig decreased CD3⁺ proliferation by 6%-20%, the combination of both agents by 64%-80%, and Hybri diminished CD3⁺ cells proliferation by 97%-99% and 94%-99% at 10 μg/mL and 20 μg/mL, respectively.

These results demonstrate that the fusion protein of the invention produces a synergistic inhibitory effect on antigen-driven lymphocyte proliferation.

### Example 3: Rat Renal Allotransplantation

A model of life-sustaining renal transplantation previously described (De Ramon et al., “CD154-CD40 T-cell co-stimulation pathway is a key mechanism in kidney ischemia-reperfusion injury”, Kidney Int., 2015, vol. 88(3), pp. 538-49) was used to test the therapeutic effects of the fusion protein. It is known that in experimental renal transplantation with highly alloreactive donor-recipient combination, severe acute rejection results in high recipients' mortality.

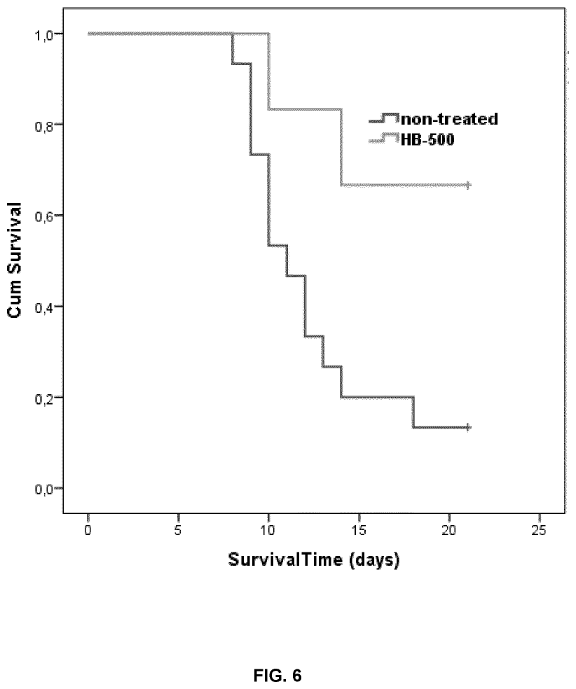

Briefly, Lewis rats were bi-nephrectomized and subsequently transplanted with a single kidney from a Wistar donor rat. Animals received an induction monotherapy immunosuppression with the fusion protein of the invention of sequence SEQ ID NO: 6 (Hybri) at 500 μg (HB-500) intraperitoneally 2 hours before transplantation, followed by the same daily dose for the next consecutive 6 days. In the control group, recipient Lewis rats received no treatment. Transplanted rats were followed for 3 weeks. In comparison with the non-treated group, rats receiving Hybri showed significantly higher survival (p<0.05) as show in FIG. 6.

These results allow concluding that the fusion protein of the invention is effective in the prevention of transplant rejection and strongly reduces the mortality associated to it.

### Example 4: Renal Ischemia-Reperfusion Injury

Ischemia-reperfusion injury (IRI) is an unavoidable phenomenon in solid organ transplantation, which may result in delayed graft function and poorer graft outcomes.

The potential protective effects of Hybri in renal ischemia-reperfusion injury were studied in Wistar rats with 40 minutes of bilateral renal ischemia followed by declamping the vascular renal pedicle (de Ramon L. et al. supra). Rats were sham operated or suffered from renal warm ischemia and were treated with the fusion protein of the invention of sequence SEQ ID NO: 6 (Hybri) at 20 mg/kg ip. 24 hours before renal ischemia; and in the control ischemic group rats received PBS.

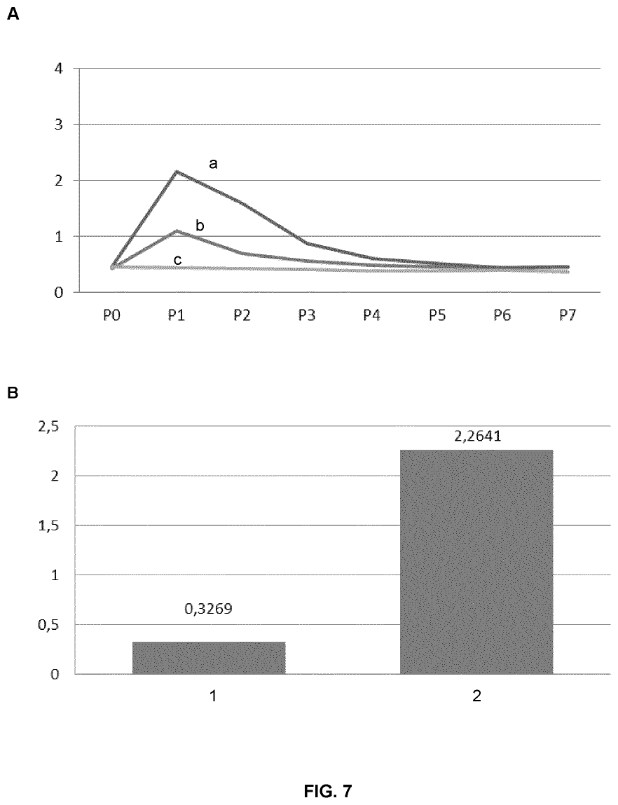

As can be seen in FIG. 7, sham operated rats treated with Hybri did not develop renal failure, and in the ischemic groups, treatment with Hybri significantly improved renal function 24 hours after warm ischemia, as can be seen by the serum creatinine levels (FIG. 7A) (P1, p=0.0038). This functional benefit was accompanied with a better preservation of renal structure. Moreover, Hybri increased the presence of T cells with regulatory phenotype (Tregs) per high power field (HPF) in renal parenchyma, as compared to non-treated ischemic kidneys (FIG. 7B).

These results demonstrate that the fusion protein of the invention is highly useful in the treatment of ischemia-reperfusion injury.

### Example 5: Murine Lupus Nephritis

NZB/W F1 hybrid mice spontaneously develop a disease closely resembling human systemic lupus erythematous (SLE) with severe renal involvement, which is the main cause of animal mortality. In fact, this model, with a well-characterized evolution of renal disease, has been widely used to study the therapeutic potential of several new agents for SLE, as previously reported (Ripoll et al., “CD40 gene silencing reduces the progression of experimental lupus nephritis modulating local milieu and systemic mechanisms”, PLoS One, 2013, vol. 8(6)).

The therapeutic effect of the fusion protein of the invention of sequence SEQ ID NO: 6 (Hybri) was studied in this mice strain and the results were compared to cyclophosphamide (CYP), which is the gold standard, or placebo (PBS). From 20 weeks of age, and close to a potential therapeutic schedule in humans, mice (n=9) received Hybri intraperitoneally (ip) at 20 mg/kg on days 0, 4, 14, 28, 56 and 84; or CYP (n=8) ip. at 50 mg/kg every 10 days; or 200 μl PBS ip. twice a week (n=8).

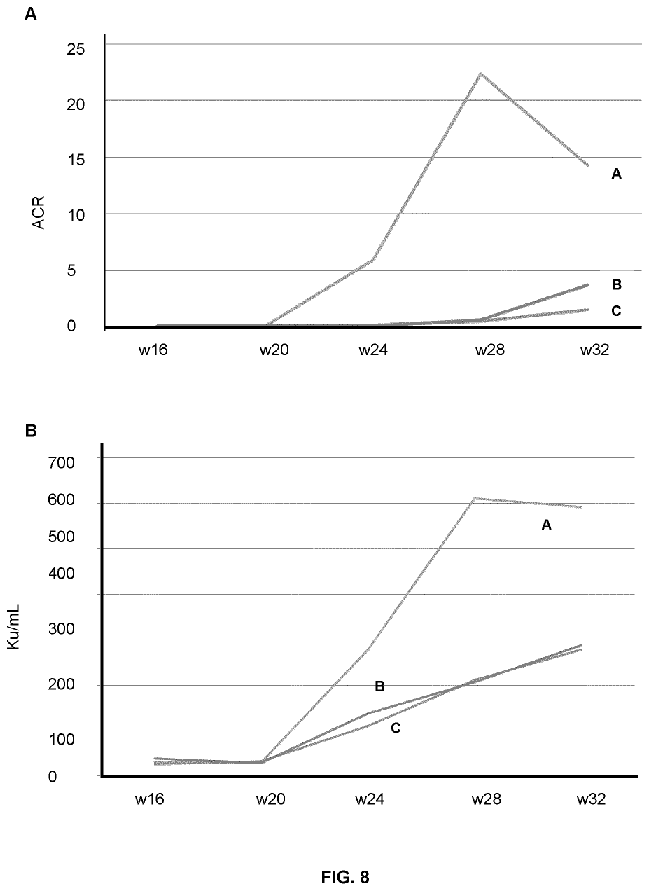

There was no mortality in the CYP and Hybri groups up to the end of experiments at 32 weeks, as compared to the control group, which presented a high mortality. Moreover, as shown in FIG. 8A, Hybri treatment was as efficient as CYP in reducing albuminuria (a marker of glomerular damage) at 3 months of therapy, while placebo treatment did not. Interestingly, Hybri effectively reduced anti-DNA antibodies to similar levels than CYP (FIG. 8B), suggesting that costimulatory signals modulation by Hybri might indirectly decrease antibody secreting cells, despite that the recombinant protein has no direct effects on B cells, in contrast to the direct effect of CYP on these cells.

These data strongly support the therapeutic efficacy of the fusion protein of the invention in lupus nephritis and other autoimmune diseases.

### CITATION LIST

Bruce S P. et al., “Update on abatacept: a selective costimulation modulator for rheumatoid arthritis”, Ann Pharmacother., 2007, vol. 41(7), pp. 1153-62
De Ramon et al., “CD154-CD40 T-cell co-stimulation pathway is a key mechanism in kidney ischemia-reperfusion injury”, Kidney Int., 2015, vol. 88(3), pp. 538-49
Ripoll et al., “CD40 gene silencing reduces the progression of experimental lupus nephritis modulating local milieu and systemic mechanisms”, PLoS One, 2013, vol. 8(6)
Levitsky J, et al., “Allospecific regulatory effects of sirolimus and tacrolimus in the human mixed lymphocyte reaction”, Transplantation, 2011, vol. 91(2), pp. 199-206
Bargalló M E. et al., “Utility of Systematic Isolation of immune cell subsets from HIV-infected individuals for miRNA profiling”, J Immunol Methods, 2017, vol. 442, pp. 12-19
Lloberas N, et al., “Dendritic cells phenotype fitting under hypoxia or lipopolysaccharide; adenosine 5′-triphosphate-binding cassette transporters far beyond an efflux pump”, Clin Exp Immunol., 2013, vol. 172(3), pp. 444-54
Latchman Y, et al. “PD-L2 is a second ligand for PD-1 and inhibits T cell activation”, Nat Immunol., 2001, vol. 2(3), pp. 261-8
Laemmli, U. K. “Cleavage of Structural Proteins during the Assembly of the Head of Bacteriophage T4”, Nature, 1970, vol. 227(5259), pp. 680-685

1. A compound comprising a fusion polypeptide of formula (I):
R1-L-R2-Fc (I)
wherein:
R1, which is at the N-terminal end of the polypeptide, is PD-L2 or a PD1-binding fragment thereof,
L is a peptide linker,
R2 is CTLA-4 or a CD80/CD86-binding fragment thereof, and
Fc, which is at the C-terminal end of the polypeptide, is an immunoglobulin Fc domain.

2. The compound according to claim 1, wherein the linker:
has a length from 5 to 50 amino acids; or, alternatively,
has at least a 50%, at least a 60%, at least a 70%, at least an 80% or at least a 90% of amino acidic residues, with respect to the total number of amino acidic residues forming the peptide linker, selected from non-polar and polar uncharged amino acids; or, alternatively,
has a length from 5 to 50 amino acids, wherein at least a 50%, at least a 60%, at least a 70%, at least an 80% or at least a 90% of the amino acids forming the peptide linker are selected from non-polar and polar uncharged amino acids; or, alternatively,
has a length from 5 to 50 amino acids, wherein at least a 50%, at least a 60%, at least a 70%, at least an 80% or at least a 90% of the amino acids forming the peptide linker are non-polar and polar uncharged amino acids; or, alternatively,
has a length from 5 to 50 amino acids, wherein at least a 50%, at least a 60%, at least a 70%, at least an 80% or at least a 90% of the amino acids forming the peptide linker are Gly and Ser; or, alternatively,
has an amino acid sequence with an identity of at least 85% with sequence SEQ ID NO: 4.

3. The compound according to claim 1, wherein:
R1 is the extracellular domain of PD-L2, R2 is the extracellular domain of CTLA-4, and the Fc comprises the hinge region, the CH2 domain, and the CH3 domain of human IgG; or alternatively,
the sequence of the fusion polypeptide has at least 85%, at least 90% or at least 95% identity with sequence SEQ ID NO: 5; or a 100% of identity with sequence SEQ ID NO: 5.

4. A dimer comprising two subunits, wherein one or both subunits correspond(s) to the compound as defined in claim 1.

5. The compound according to claim 1 or a dimer comprising two subunits wherein one or both subunits correspond(s) to the compound, wherein the compound further comprises a heterologous moiety.

6. A polynucleotide which encodes the compound as defined in claim 1 or a dimer comprising two subunits wherein one or both subunits correspond(s) to the compound.

7. A vector comprising the polynucleotide as defined in claim 6.

8. A host cell which is transformed or transfected with the polynucleotide as defined in claim 6 or a vector comprising the polynucleotide.

9. A cell culture comprising the host cell as defined in claim 8.

10. A process for the production of a compound as defined in claim 1, comprising:
(a) culturing a host cell transformed or transfected with a polynucleotide which encodes the compound; or, alternatively,
(b) in vitro transcription and/or translation of the polynucleotide to express the compound; and
(c) isolating the expressed compound.

11. A pharmaceutical composition comprising a therapeutically effective amount of the compound as defined in claim 1 or a dimer comprising two subunits wherein one or both subunits correspond(s) to the compound wherein the pharmaceutical composition further comprises at least one pharmaceutically acceptable carrier or excipient.

12. A kit comprising:
the compound as defined in claim 1; or
a dimer comprising two subunits wherein one or both subunits correspond(s) to the compound; or
a pharmaceutical composition comprising: a therapeutically effective amount of the compound or a dimer comprising two subunits wherein at least one of the two subunits is one or both subunits correspond(s) to the compound, the pharmaceutical composition further comprising at least one pharmaceutically acceptable carrier or excipient; and
optionally, instructions for its use.

13. (canceled)

14. (canceled)

15. A method for treating and/or preventing a disease selected from an autoimmune disease and transplant rejection, the method comprising administering to a subject in need thereof, a therapeutically effective amount of:
a compound as defined in claim 1; or
a dimer comprising two subunits wherein one or both subunits correspond(s) to the compound; or
a pharmaceutical composition comprising: a therapeutically effective amount of the compound or a dimer comprising two subunits wherein one or both subunits correspond(s) to the compound, wherein the pharmaceutical composition further comprises at least one pharmaceutically acceptable carrier or excipient.

16. A method for inhibiting T cell activation in an isolated biological sample from a subject, the method comprising the step of contacting the isolated biological sample with:
a compound as defined in claim 1; or
a dimer comprising two subunits, wherein one or both subunits correspond(s) to the compound; or
a pharmaceutical composition comprising: a therapeutically effective amount of the compound or a dimer comprising two subunits wherein one or both subunits correspond(s) to the compound, wherein the pharmaceutical composition further comprises at least one pharmaceutically acceptable carrier or excipient.

17. A method of transplantation of a mammalian organ or tissue, the method comprising:
removing the organ or tissue from a donor;
contacting the organ or tissue with:
a compound as defined in claim 1; or
a dimer comprising two subunits wherein one or both subunits correspond(s) to the compound; or
a pharmaceutical composition comprising:
a therapeutically effective amount of the compound or a dimer comprising two subunits wherein one or both subunits correspond(s) to the compound, wherein the pharmaceutical composition further comprises at least one pharmaceutically acceptable carrier or excipient; and
transplanting said organ or tissue in the recipient.
